(19) United States
(12) Patent Application Publication
(10) Pub. No.: US 2025/0195920 A1
(43) Pub. Date: Jun. 19, 2025

(54) INFORMATION PROCESSING APPARATUS, DISPLAY DEVICE, RADIATION THERAPY PLANNING APPARATUS, INFORMATION PROCESSING METHOD, AND PROGRAM

(71) Applicant: TOHOKU UNIVERSITY, Miyagi (JP)

(72) Inventors: Yoshiyuki KATSUTA, Miyagi (JP); Noriyuki KADOYA, Miyagi (JP); Tomohiro KAJIKAWA, Kyoto (JP); Keiichi JINGU, Miyagi (JP)

(73) Assignee: TOHOKU UNIVERSITY, Miyagi (JP)

(21) Appl. No.: 18/976,294

(22) Filed: Dec. 10, 2024

(30) Foreign Application Priority Data
Dec. 13, 2023 (JP) 2023-210514

(51) Int. Cl. A61N 5/10

(52) U.S. Cl. CPC A61N 5/1031; A61N 5/1049

(57) ABSTRACT

For example, a novel method capable of supporting diagnosis, treatment, and the like in consideration of a side effect of the radiation therapy is proposed. An information processing apparatus includes a calculation unit configured to calculate a first value corresponding to position information based on a model and mathematical information, the model being generated using training data regarding a plurality of subjects and configured to calculate the first value regarding a side effect of radiation therapy, the mathematical information being regarding an organ of a first subject and corresponding to the position information.

### CROSS-REFERENCE TO RELATED APPLICATION

The present application claims priority under 35 U.S.C § 119 (a) to Japanese Patent Application No. 2023-210514 filed on Dec. 13, 2023, which is hereby expressly incorporated by reference, in its entirety, into the present application.

### BACKGROUND OF THE INVENTION

### 1. Field of the Invention

The present invention relates to an information processing apparatus and the like.

### 2. Description of Related Art

In the related art, there is a technique (radiation therapy) of treating a tumor such as lung cancer (for example, non-small cell lung cancer (NSCLC)) using a radiation therapy (for example, PTL 1). However, as a side effect (or regarded as an adverse event) of the radiation therapy, for example, it is known that radiation pneumonia (hereinafter, appropriately referred to as “radiation pneumonitis (RP)”) may occur.

### CITATION LIST

### Patent Literature

PTL 1: JP2023-049895A

### SUMMARY OF THE INVENTION

For example, when treating the lung cancer or the like using the radiation therapy, various healthy structures such as lung tissue, esophagus, and trachea may be close to a tumor site (tumor region), and the treatment may be difficult. For example, the RP is highly risky and occurs frequently, and is often fatal to a patient. However, preventive methods in the related art are not sufficient and it is far from overcoming the RP.

The invention has been made in view of such a problem, and an object thereof is to propose a novel method capable of supporting diagnosis, treatment, and the like in consideration of, for example, a side effect of radiation therapy.

According to a first aspect of the invention, an information processing apparatus includes a calculation unit configured to calculate a first value corresponding to position information based on a model and a second value, the model being generated using training data regarding a plurality of subjects and configured to calculate the first value regarding a side effect of radiation therapy, the second value being based on mathematical information regarding an organ of a first subject and corresponding to the position information.

According to a second aspect of the invention, an information processing method includes calculating a first value corresponding to position information based on a model and a second value, the model being generated using training data regarding a plurality of subjects and configured to calculate the first value regarding a side effect of radiation therapy, the second value being based on mathematical information regarding an organ of a first subject and corresponding to the position information.

According to a third aspect of the invention, a program to be executed by a computer causes a computer to execute calculating a first value corresponding to position information based on a model and a second value, the model being generated using training data regarding a plurality of subjects and configured to calculate the first value regarding a side effect of radiation therapy, the second value being based on mathematical information regarding an organ of a first subject and corresponding to the position information.

According to the invention, it is possible to appropriately support diagnosis, treatment, and the like in consideration of, for example, a side effect of radiation therapy.

### BRIEF DESCRIPTION OF THE DRAWINGS

FIG. 1 is a diagram illustrating an example of an information processing system.

FIG. 2 is a diagram illustrating an IMRT.

FIG. 3 is a block diagram illustrating training of a pneumonia occurrence prediction model.

FIG. 4 is a diagram illustrating the pneumonia occurrence prediction model.

FIG. 5 is a block diagram illustrating inference of a pneumonia occurrence susceptibility level.

FIG. 6 is a diagram illustrating a flow of generation of a pneumonia occurrence hazard map.

FIG. 7 is a diagram illustrating an example of a two-dimensional pneumonia occurrence hazard map.

FIG. 8 is a diagram illustrating an example of a three-dimensional pneumonia occurrence hazard map.

FIG. 9 is a diagram illustrating another example of the information processing system.

FIG. 10 is a flowchart illustrating an example of a flow of processing executed by a processing unit of an information processing apparatus.

FIG. 11 is a flowchart illustrating an example of a flow of processing executed by the processing unit of the information processing apparatus.

FIG. 12 is a diagram illustrating an example of an evaluation result of a model.

FIG. 13 is a diagram illustrating an example of an evaluation result of a model.

FIG. 14 is a diagram illustrating an example of a radiation therapy planning apparatus.

FIG. 15 is a diagram illustrating a radiation therapy plan.

### DESCRIPTION OF EMBODIMENTS

Hereinafter, an exemplary embodiment of the invention will be described with reference to the drawings.

In the description of the drawings, the same elements are denoted by the same reference signs, and a repeated description may be omitted.

The constituent elements described in the embodiment are merely examples, and are not intended to limit the scope of the invention.

### EMBODIMENT

Hereinafter, an exemplary embodiment for implementing an information processing technique of the invention will be described.

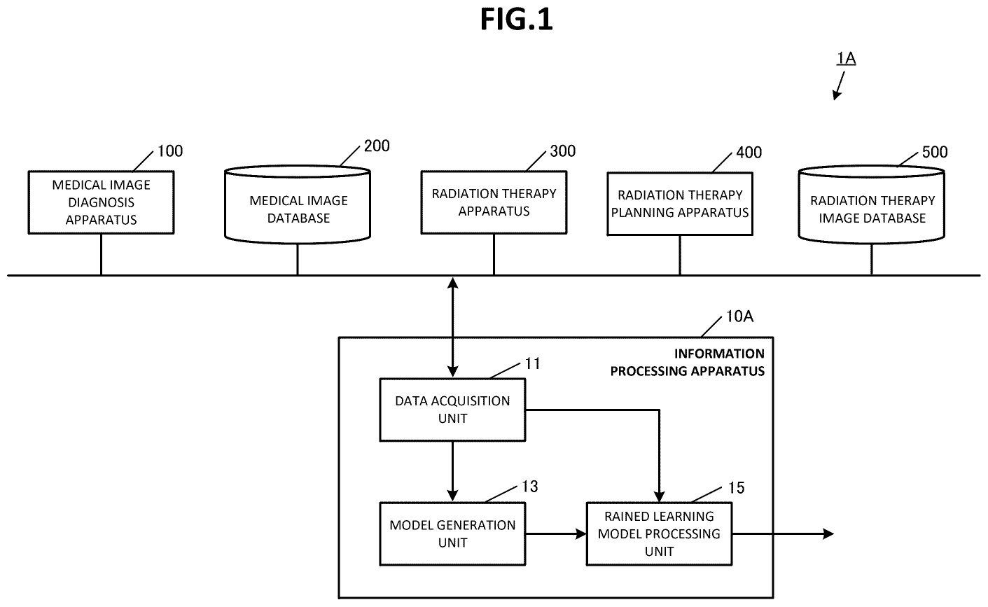

FIG. 1 is a diagram illustrating an example of a configuration of an information processing system 1A according to an aspect of the embodiment.

The information processing system 1A includes, for example, a medical image diagnosis apparatus 100, a medical image database 200, a radiation therapy apparatus 300, a radiation therapy planning apparatus a radiation therapy image database 500, and an information processing apparatus 10A as an example of an information processing apparatus 10.

These devices may be capable of communicating with each other via, for example, a bus or a communication unit. A communication mode of the communication unit may be wired communication or wireless communication.

The medical image diagnosis apparatus 100 may include, for example, apparatuses such as a simple X-ray apparatus, an X-ray computed tomography (CT) apparatus, and a magnetic resonance imaging (MRI) apparatus for acquiring and evaluating morphological information (information on an anatomical structure) and apparatuses (nuclear medicine exanimation apparatuses) such as a positron emission tomography (PET) apparatus and a single photon emission computed tomography (SPECT) apparatus for acquiring and evaluating function information (physiological function information). The medical image diagnosis apparatus may be any of the various devices, or may be implemented by a combination of the various devices. A PET-CT apparatus, a SPECT-CT apparatus, or the like may be used.

Medical image data (for example, volume data or image data obtained by converting the volume data into an image) by the medical image diagnosis apparatus 100 may be stored in the medical image database 200 in association with identification information (ID, name, and the like) of a person (hereinafter, may be inclusively referred to as an “examinee”) such as an examinee or a patient and information on date and the like.

A person may be an example of a subject. In the present specification, the subject may be subject to medical practices such as a radiation therapy.

The medical image data may include morphological information data and function information data.

The medical image database 200 may be provided in the information processing apparatus 10A.

The radiation therapy apparatus 300 is an apparatus for performing radiation therapy, and may be, for example, an apparatus for intensity modulated radiation therapy (IMRT) including TomoTherapy or a multi-leaf collimator (MLC) for performing the IMRT. The IMRT may include volumetric modulated arc therapy (VMAT).

However, the invention is not limited thereto, and for example, an apparatus for performing three-dimensional conformal radiation therapy (3D-CRT) or stereotactic radiation therapy (SRT) may be applied. Alternatively, these devices may be combined.

As the radiation therapy, therapy using an X-ray or γ-ray classified as electromagnetic waves may be applied, or therapy using an α-ray, a β-ray, an electron beam, a proton beam, a heavy particle beam, or a neutron beam classified as a particle beam may be applied. For example, proton beam therapy, heavy particle beam therapy, and neutron beam therapy may be applied, and an apparatus thereof may be applied as the radiation therapy apparatus 300. Any combination of the above may be applied.

The radiation therapy planning apparatus 400 may be, for example, a computer apparatus (information processing apparatus) configured to create (generate) a radiation therapy plan that is a plan of radiation therapy to be performed by the radiation therapy apparatus 300. The radiation therapy planning apparatus may be referred to as a radiation therapy plan creation apparatus (radiation therapy plan generation apparatus).

For example, data regarding a radiation therapy plan (for example, irradiation data regarding radiation or image data obtained by converting the irradiation data into an image) created by the radiation therapy planning apparatus 400 may be stored in the radiation therapy image database 500 as radiation therapy image data.

The radiation therapy image database 500 may be provided in the radiation therapy planning apparatus 400 or the information processing apparatus 10A.

The information processing apparatus 10A is a computer apparatus including, for example, a data acquisition unit 11, a model generation unit 13, and a trained learning model processing unit 15 as functional units, and performs various types of processing based on data regarding a medical image acquired by the data acquisition unit 11 and stored in the medical image database 200, data regarding a radiation therapy image acquired by the data acquisition unit 11 and stored in the radiation therapy image database 500, and the like.

These functional units may be, for example, functional units (functional blocks) provided in a processing unit (processing device, not illustrated) or a control unit (control device, not illustrated) of the information processing apparatus 10A, and may include a processing circuit such as a central processing unit (CPU), a graphics processing unit (GPU), a digital signal processor (DSP), an application specific integrated circuit (ASIC), or a field programmable gate array (FPGA).

Based on the data acquired by the data acquisition unit 11, the model generation unit 13 generates, for example, a model (hereinafter, appropriately referred to as a “pneumonia occurrence prediction model”) for predicting the occurrence of pneumonia (for example, RP) accompanying radiation therapy based on machine learning (for example, supervised learning).

The trained learning model processing unit 15 is a processing unit of a trained pneumonia occurrence prediction model (hereinafter, appropriately referred to as a “trained pneumonia occurrence prediction model”) generated by the model generation unit 13, and calculates (infers) a predetermined physical quantity related to the occurrence of pneumonia using the trained pneumonia occurrence prediction model.

The calculated physical quantity may be output from the trained learning model processing unit 15.

Here, the “output” of information and data (information and the like) may include, for example, concepts of output t (internal output) of information to another functional unit in the own device, and output (external output), transmission (external transmission), display, and sound output of information to a device (external device) other than the own device.

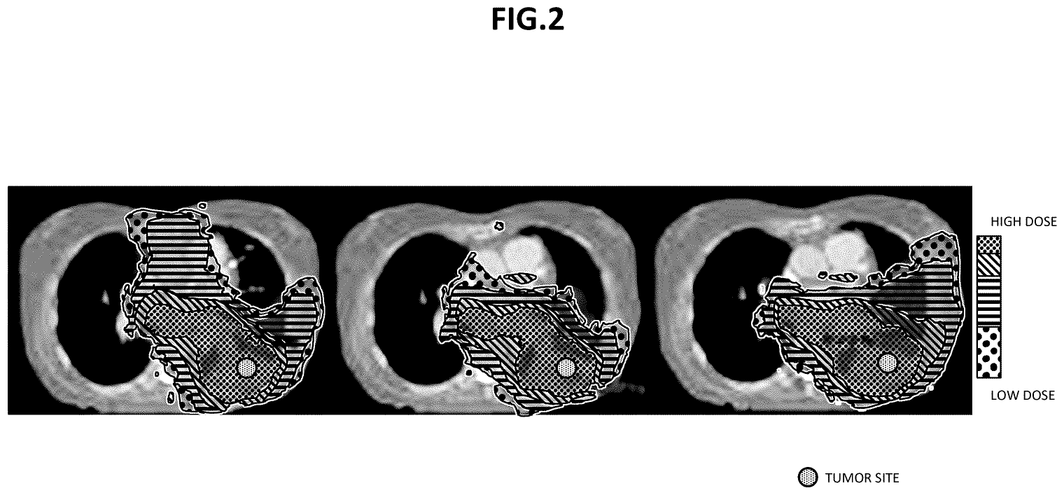

FIG. 2 is a diagram illustrating an outline of the IMRT.

In the IMRT, a specific site may or may not be irradiated with radiation, and an irradiation range (distribution, spread) thereof can be minutely adjusted by, for example, the radiation therapy planning apparatus 400 to create a radiation therapy plan.

For example, when a site (region) indicated by a hatched circle is a tumor site of lung cancer, the irradiation range can be set in various patterns as shown in the drawing, for example.

Note that a difference (fluctuation) in radiation dose is roughly indicated by four different hatchings.

As a pneumonia prevention method in radiation therapy, for example, a method of simply reducing radiation exposure of a lung tissue by using a CT image and a method of using a function image (lung function image) in which the oxygen-carbon dioxide exchange efficiency is visualized and preferentially reducing radiation exposure of a lung tissue with high exchange efficiency are conceivable.

The radiation therapy using a function image can be performed using, for example, a nuclear medicine image and a CT image, but there are problems that there is little evidence that damage to high-functioning lung regions (high-functioning regions) is equivalent to the occurrence of pneumonia, and that a plurality of functional regions cannot be used in an integrated manner.

In the embodiment, as an example, a pneumonia occurrence prediction model capable of predicting the likelihood (susceptibility) of occurrence of pneumonia as a side effect of the radiation therapy by a numerical value is generated.

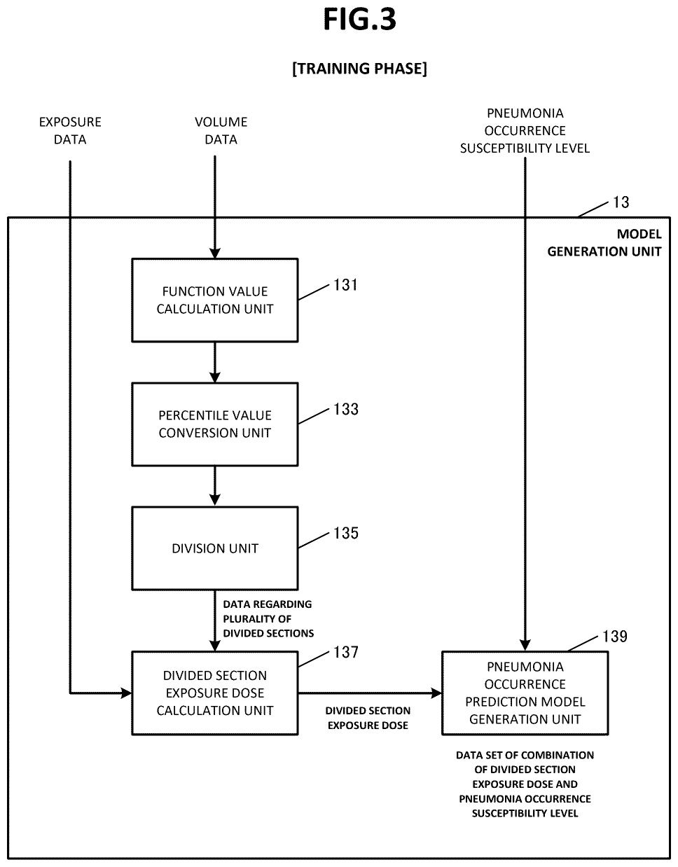

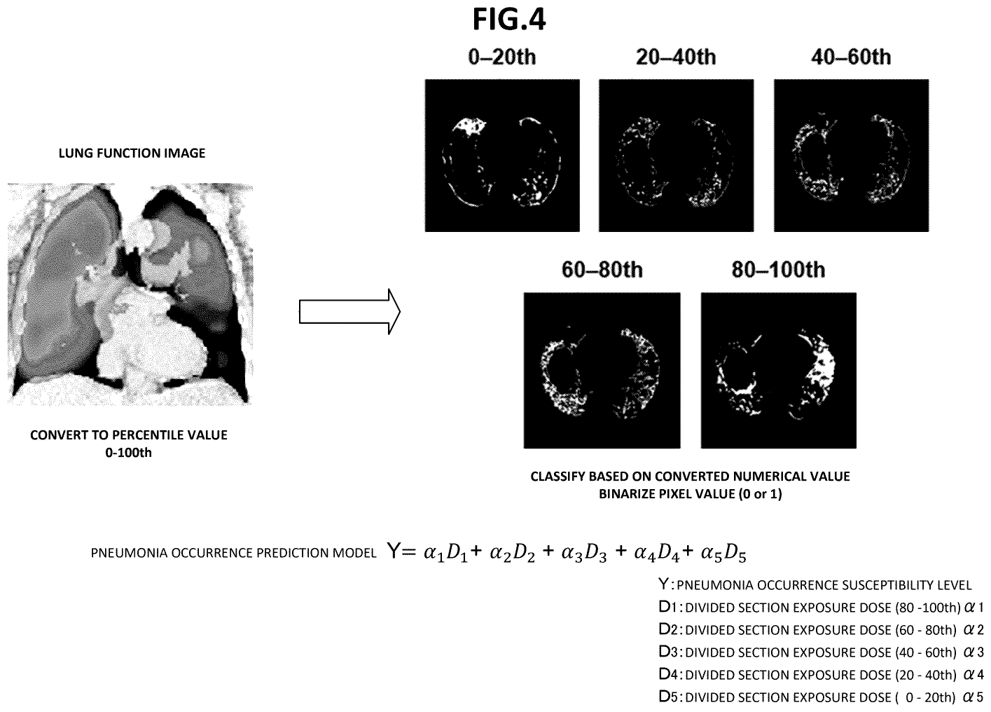

FIGS. 3 and 4 are diagrams illustrating principles of generation and the like of a pneumonia occurrence prediction model according to the embodiment.

Here, as an example, a case where 4D CT data is acquired by the data acquisition unit 11 will be exemplified. The 4D CT data is, for example, three-dimensional volume data in time series, and is dynamic data according to respiration of an examinee. Specifically, for example, the data may be data including 3D CT data at two time-points of an exhalation position (peak exhalation position or the like) and an inhalation position (peak inhalation position) of the examinee.

When CT is applied, processing described below may be performed based on not only the 4D CT data but also the 3D CT data and 2D CT data.

### (1) Training Phase

As illustrated in FIG. 3, the model generation unit 13 includes, for example, a function value calculation unit 131, a percentile value conversion unit 133, a division unit 135, a divided section exposure dose calculation unit 137, and a pneumonia occurrence prediction model generation unit 139 as functional units.

### (A) Quantification of Function Value

For example, the function value calculation unit 131 performs deformable image registration (DIR) and quantitative evaluation (quantitative analysis) on the 4D CT data (4D CT image) acquired by the data acquisition unit 11 to generate lung function data (lung function image) obtained by quantifying pulmonary ventilation.

The generated lung function data (lung function image) may be stored in the information processing apparatus 10A or a database (not illustrated) outside the information processing apparatus 10A.

In this case, as a method of quantitative evaluation, for example, a method according to Hounsfield Unit (HU) may be applied, and for example, the following formula (1) may be used.

$$\frac{{V}_{i n} - {V}_{ex}}{{V}_{ex}} = 1000 \frac{H {U}_{i n} - {HU}_{ex}}{{HU}_{ex} ( 1000 + {HU}_{i n} )} \qquad ( 1 )$$

However, “Vᵢₙ” and “Vₑₓ” represent a volume at the time of peak inhalation (peak inhalation image) and a volume at the time of peak exhalation (peak exhalation image) respectively, and “HUᵢₙ” and “HUₑₓ” represent corresponding HU values.

As a method of quantitative evaluation, for example, a method of analyzing a degree of change in the volume of the lungs from exhalation to inhalation by using a Jacobian matrix may be used.

In the following processing, lung function information (for example, a lung function value) may be used, and an acquisition method thereof is not limited. For example, a lung function value (lung function image) acquired based on the above-described nuclear medicine examination may be used.

### (B) Percentile Value Conversion Processing

The percentile value conversion unit 133 performs percentile value conversion processing of converting a lung function value (hereinafter, may be simply referred to as a “function value”) quantified in the processing of (A) to a percentile value (for example, 0 to 100). The percentile value obtained in this manner is referred to as a “function percentile value” for convenience.

A functional unit configured to perform noise reduction processing (noise removal processing) on the function value (function image: 4D CT ventilation image constituted by function values quantified by HU, for example) quantified in the processing of (A) may be configured in the model generation unit 13. Specifically, for example, a filter processing unit configured to perform filter processing using a median filter (for example, a median filter with a width of 3×3×3 voxels) may be configured in the model generation unit 13.

The filter processing unit may perform filter processing using a smoothing filter such as a moving average filter or a Gaussian filter.

### (C) Division Processing

The division unit 135 divides data (image), in which a position and the function percentile value are associated with each other, into a plurality of pieces of data (a plurality of images), based on a preset upper limit threshold (cutoff upper limit) and a lower limit threshold (cutoff lower limit) for the function percentile value obtained in the processing (B) (or the filter processing). The threshold may be set automatically by the information processing apparatus 10 (automatic setting) or may be set by the information processing apparatus 10 based on a user input (manual setting). The same applies to various settings.

Specifically, for example, the cutoff lower limit is set from 0th to the 80th for each interval of 20, and the cutoff upper limit is set from the 20th to the 100th for each interval of 20. A function percentile value (a function percentile value associated with a position) included in a section defined by the cutoff lower limit and the cutoff upper limit is extracted. Accordingly, in this example, data in which the position and the function percentile value are associated, corresponding to five sections of “0-20th”, “20-40th”, “40-60th”, “60-80th”, and “80-100th”, is obtained. For convenience, each of the divided sections is referred to as a “divided section”, and the number of the divided sections is referred to as a “division number”.

For example, based on the data (in the above example, five pieces of data, that is, five function percentile images) obtained in the processing of (C) in which the position and the function percentile value are associated with each other, the divided section exposure dose calculation unit 137 calculates an exposure dose (hereinafter, referred to as a “divided section exposure dose”) corresponding to each divided section.

Specifically, for example, by performing segmentation of an image, region division is performed on the divided section, and an exposure dose for each region (for example, an average dose obtained by averaging the exposure dose for each region) is calculated.

A ratio (for example, V20 Gy) of a lung volume exposed over a specific exposure dose (a set threshold dose: 20 Gy (gray)) with respect to the whole lung may be calculated for each divided section. The calculated ratio of the lung volume may be output. The threshold dose may be set to any value (5 Gy, 40 Gy, or the like).

In this case, for example, the exposure dose for each region may be calculated by superimposing a region divided image and an image of the radiation therapy plan by using data regarding the radiation therapy plan (also referred to as data regarding exposure information (exposure data)) including an exposure dose in calculation created by the radiation therapy planning apparatus 400. However, the exposure dose calculated in this manner is merely an estimated exposure dose (estimated exposure dose) in calculation.

In this example, since the exposure dose is calculated for each region, a pneumonia occurrence susceptibility level to be described later is also a value for each region.

An average of exposure doses at positions where the same percentile value, etc. may be used for each of the divided sections.

An exposure dose calculated for each position may be used for each of the divided sections.

Hereinafter, for the sake of convenience, a divided section exposure dose corresponding to the divided section of “80-100th” is denoted as D₁,
a divided section exposure dose corresponding to the divided section “60-80th” is denoted as D₂,
a divided section exposure dose corresponding to the divided section of “40-60th” is denoted as D₃,
a divided section exposure dose corresponding to the divided section of “20-40th” is denoted as D₄, and
a divided section exposure dose corresponding to the divided section of “0-20th” is denoted as D₅.

The pneumonia occurrence prediction model generation unit 139 generates, for example, a pneumonia occurrence prediction model for inferring a value (hereinafter referred to as a “pneumonia occurrence susceptibility level”) indicating the likelihood (susceptibility) of occurrence of pneumonia accompanying radiation therapy. The pneumonia occurrence susceptibility level may be an example of a first value related to a side effect of the radiation therapy, and may be defined as a unitless value equal to or greater than 0.

Specifically, for example, a pneumonia occurrence prediction model is generated by machine learning using a plurality of data sets in which a combination of the divided section exposure dose calculated by the divided section exposure dose calculation unit 137 and the pneumonia occurrence susceptibility level acquired in advance as epidemiological data relating to occurrence and non-occurrence of pneumonia (side effect) is set as one data set. The pneumonia occurrence prediction model generated in this manner is referred to as a “trained pneumonia occurrence prediction model”.

In the embodiment, a pneumonia occurrence prediction model for training the pneumonia occurrence prediction model generation unit 139 is, for example, a model represented by the following formula (2).

$$Y = {α}_{1} {D}_{1} + {α}_{2} {D}_{2} + {α}_{3} {D}_{3} + {α}_{4} {D}_{4} + {α}_{5} {D}_{5} \qquad ( 2 )$$

Here, “α₁” to “α₅” are weights (regression coefficients) corresponding to the divided section exposure doses “D₁” to “D₅”, respectively, and Y is the pneumonia occurrence susceptibility level. That is, the pneumonia occurrence prediction model is defined as a model in which the pneumonia occurrence susceptibility level is represented by a weighted sum of the divided section exposure doses based on the regression coefficients.

The divided section exposure dose corresponds to an explanatory variable, and the pneumonia occurrence susceptibility level corresponds to an objective variable.

In a training phase, a combination of the divided section exposure dose of each divided section calculated by the divided section exposure dose calculation unit 137 and the pneumonia occurrence susceptibility level may be used as one piece of data, and the training may be performed by using, for example, data (data set) regarding a plurality of examinees as training data.

The training data may be referred to as data for training.

Differently, the training data may be data including verification data. The verification data may be, for example, data for confirming evaluation of a model based on a score.

In the embodiment, the pneumonia occurrence prediction model generation unit 139 generates a pneumonia occurrence prediction model based on, for example, Lasso regression.

The Lasso regression is a method of simultaneously performing the determination of the regression coefficient “α” and feature selection based on an objective function to which the L1 norm is introduced. A hyperparameter (λ of an L1 regularization term) representing the magnitude of penalty and the above regression coefficients “α₁” to “α₅” can be calculated using the above training data. Since the Lasso regression itself is known, a detailed description thereof will be omitted.

One reason for using the Lasso regression is to eliminate an unnecessary feature by feature selection. The Lasso regression is more effective when the division number is increased.

Differently from the embodiment, for example, a multiple regression analysis method such as Ridge regression, Elastic Net regression, Bayesian linear regression, robust regression, a most likelihood estimation method, or a gradient descent method may be used. In addition, for example, a technique of machine learning such as deep learning and multi-layer perceptron (MLP) (here, deep learning and MLP are types of machine learning), which are developed forms of a neural network, may be applied. A genetic algorithm may be used.

FIG. 4 is a diagram illustrating, using images, a flow of training of the model described with reference to FIG. 3. As described above, actually, the above processing can be performed using three-dimensional volume data. Here, the description is made using two-dimensional images for easy understanding.

A left portion of FIG. 4 illustrates an example of a lung function image (4D CT ventilation image) of a certain examinee.

Five divided images obtained by performing the above-described processing on the function image are illustrated at the right portion of FIG. 4. In FIG. 4, divided images for which classification is performed based on the function percentile value and a pixel value is binarized are shown.

For example, a region indicated in white in the image of “80-100th” indicates a region with a high function value, that is, a high-functioning region.

From the viewpoint of an image, training of the model is performed by using a plurality of data sets of data regarding a combination of the divided section exposure doses “D₁” to “D₅”, which are calculated based on the superposition of the respective divided images and an image of the IMRT, and the pneumonia occurrence susceptibility level “Y” based on, for example, the above-described epidemiological data.

### (2) Inference Phase

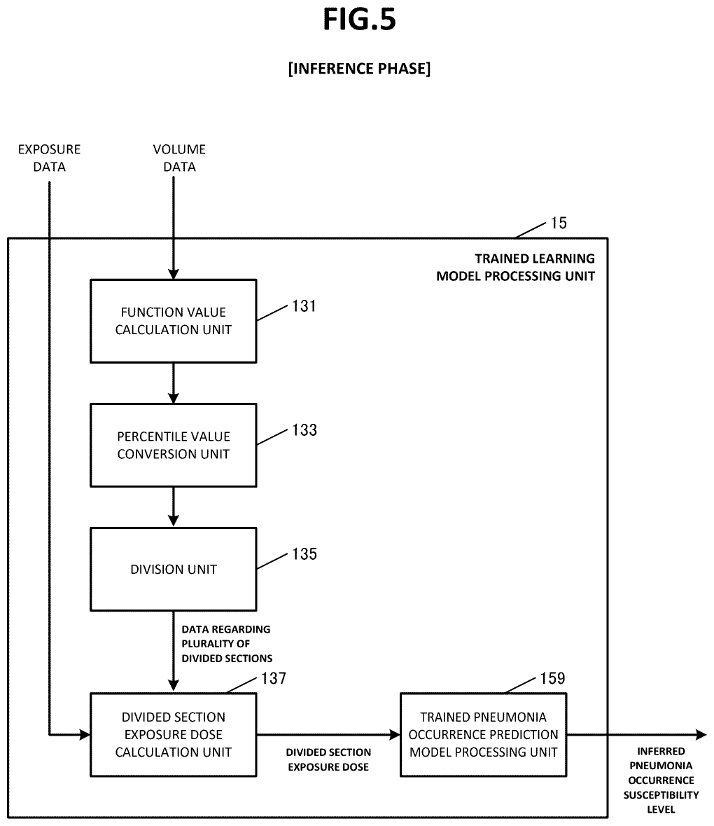

As illustrated in FIG. 5, the trained learning model processing unit 15 includes, for example, the function value calculation 131, unit the percentile value conversion unit 133, the division unit 135, the divided section exposure dose calculation unit 137, and a trained pneumonia occurrence prediction model processing unit 159 as functional units.

The function value calculation unit 131, the percentile value conversion unit 133, the division unit 135, and the divided section exposure dose calculation unit 137 may be the same as those in FIG. 3.

The trained pneumonia occurrence prediction model processing unit 159 infers the pneumonia occurrence susceptibility level according to formula (2) by using the divided section exposure doses “D₁” to “D₅” calculated by the divided section exposure dose calculation unit 137 and the regression coefficients “α₁” to “α₅” obtained in the training phase. The pneumonia occurrence susceptibility level that is inferred is referred to as an “inferred pneumonia occurrence susceptibility level”.

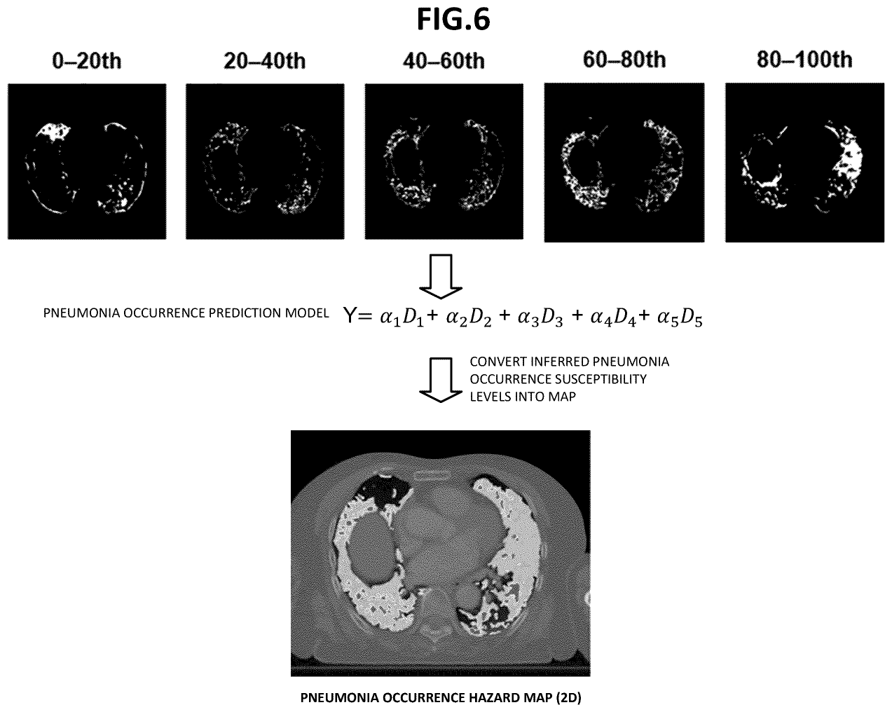

FIG. 6 is a diagram schematically illustrating, using images, a flow of inferring the pneumonia occurrence susceptibility level.

In an upper portion, binarized images for the respective divided sections shown in FIG. 4 are shown.

According to the same method as that described above, the divided section exposure doses “D₁” to “D₅” of the respective divided sections are calculated. Then, using the calculated divided section exposure doses “D₁” to “D₅” and the regression coefficients “α” to “α₅” obtained in the training phase, the pneumonia occurrence susceptibility level is inferred according to formula (2).

Based on the association between the position and the inferred pneumonia occurrence susceptibility level, the inferred pneumonia occurrence susceptibility levels are converted into an image (visualized as an image) in a map format, and the image is referred to as a “pneumonia occurrence hazard map”.

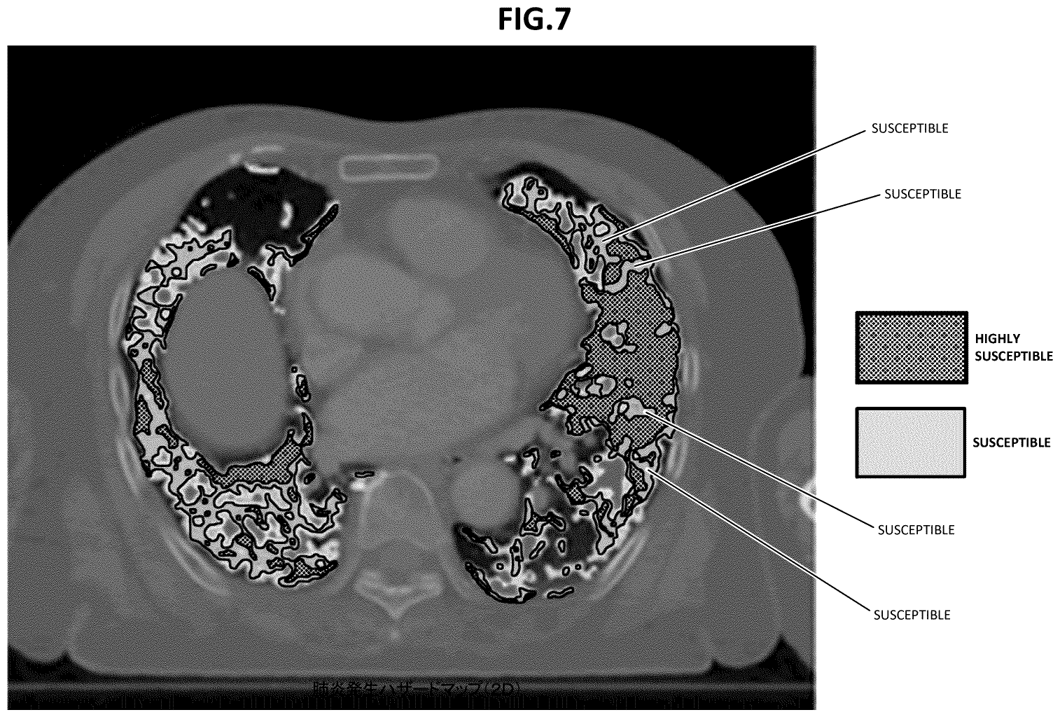

In a lower portion of FIG. 6, a two-dimensional pneumonia occurrence hazard map corresponding to the image shown in FIG. 4 is shown. FIG. 7 shows an enlarged view of the two-dimensional pneumonia occurrence hazard map.

In the two-dimensional pneumonia occurrence hazard map in FIG. 7, a hatched region of dots indicates a region with the highest susceptibility to occurrence of pneumonia, for example, a “very susceptible” region (whose susceptibility is very high). In addition, a region in light gray whose contour is drawn indicates a region with the second-highest susceptibility to occurrence of pneumonia, for example, a “susceptible” region (whose susceptibility is high). To facilitate understanding, facing the drawing, some “susceptible” regions in a lung region on the right side are indicated as “susceptible” by leader lines. A “very susceptible” region (whose susceptibility is very high) may be considered to represent a so-called red zone, and a “susceptible” region (whose susceptibility is high) may be considered to represent a so-called yellow zone.

Actually, for each of the two types of regions, a plurality of regions corresponding to the susceptibility are present.

In view of the hazard map, the susceptibility may be classified into a plurality of levels based on, for example, the inferred pneumonia occurrence susceptibility level.

For example, in the embodiment, since the division number is “5”, the susceptibility may be classified into five levels accordingly. For example, the susceptibility may be classified into “very high susceptibility”, “high susceptibility”, “medium susceptibility”, “low susceptibility”, and “very low susceptibility” in descending order of susceptibility.

In this case, for example, thresholds of different levels may be set as thresholds (cutoff value) for the inferred pneumonia occurrence susceptibility level, and the susceptibility may be classified by performing threshold determination with respect to the inferred pneumonia occurrence susceptibility level.

The setting of the number of levels of susceptibility may be appropriately changed.

In this case, after colors corresponding to classified susceptibility are set, a pneumonia occurrence hazard map in which regions are color-coded may be generated and displayed.

For example, in the case of the five levels described above, a pneumonia occurrence hazard map in which “very high susceptibility=red”, “high susceptibility=orange”, “medium susceptibility=yellow”, “low susceptibility=green”, and “very low susceptibility=blue” may be generated and displayed.

The susceptibility to occurrence of pneumonia may be considered to be represented by the regression coefficient “α”. This is because the region is divided for each of the divided sections, and the divided section exposure dose is calculated for each region.

A region classified to the divided region of “α=0” may be considered as a region not involved in the occurrence of pneumonia, in other words, a “safe” region.

As will be described later, in the above example, the regression coefficient “α₅” of the divided section “0-20th” and the regression coefficient “α₄” of the divided section “20-40th” are “0” by using the Lasso regression in the embodiment. Therefore, a region classified to “0-20th” and a region classified to “20-40th” may be considered as “safe” regions.

However, in a case where there is no large difference in value of the regression coefficient “α” corresponding to the divided sections, it cannot be said that the regions classified to the divided section are necessarily “safe” just because the calculated regression coefficient “α” is small. However, it may be considered that the region classified to the divided section of “α=0” is a region not involved in the occurrence of pneumonia.

The criterion for “safe” can be appropriately set and changed. For example, a region whose inferred pneumonia occurrence susceptibility level is less than (or equal to or less than) a smallest threshold among the set thresholds of different levels may be set as “safe”.

Instead of color coding, the regions may be distinguished and displayed with different hatching.

Alternatively, the regions may be displayed in any of monochrome, black and white, gray scale, and color.

In addition, data in which the position or the region and the inferred pneumonia occurrence susceptibility level (numerical value) are associated may be displayed in the form of, for example, a chart or a table.

Further, based on a user input such as clicking or touching (in case of a touch screen) any position or region in the pneumonia occurrence hazard map displayed on a display device 600, control of displaying a corresponding inferred pneumonia occurrence susceptibility level (numerical value) of the position or region is performed, whereby the user may confirm detailed data.

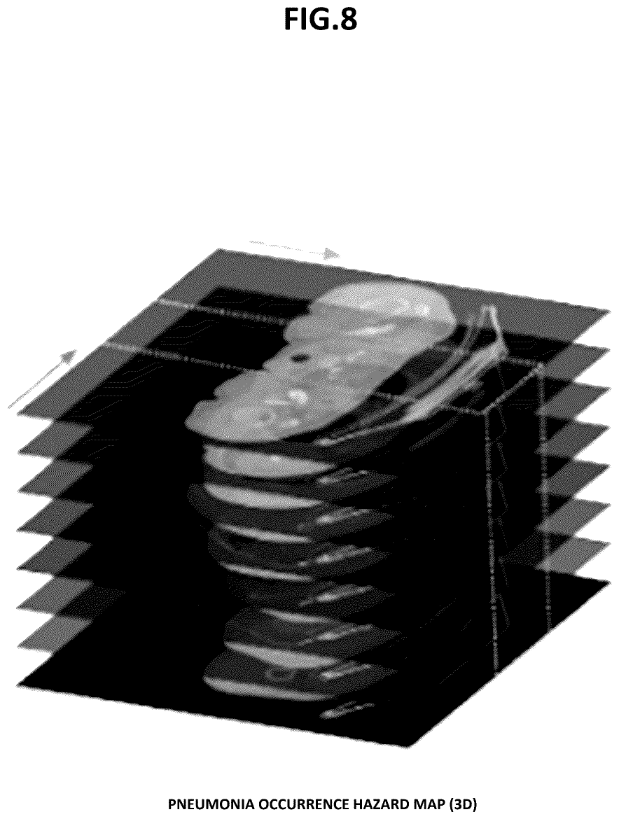

In the embodiment, since the above processing is performed using three-dimensional volume data, the pneumonia occurrence hazard map is generated, for example, as a three-dimensional map as shown in FIG. 8. Similarly, three-dimensional lung function data is obtained by using 4D CT data.

In this case, control of displaying a map in any different viewpoint or cross section on the display device 600 may be performed based on the user input.

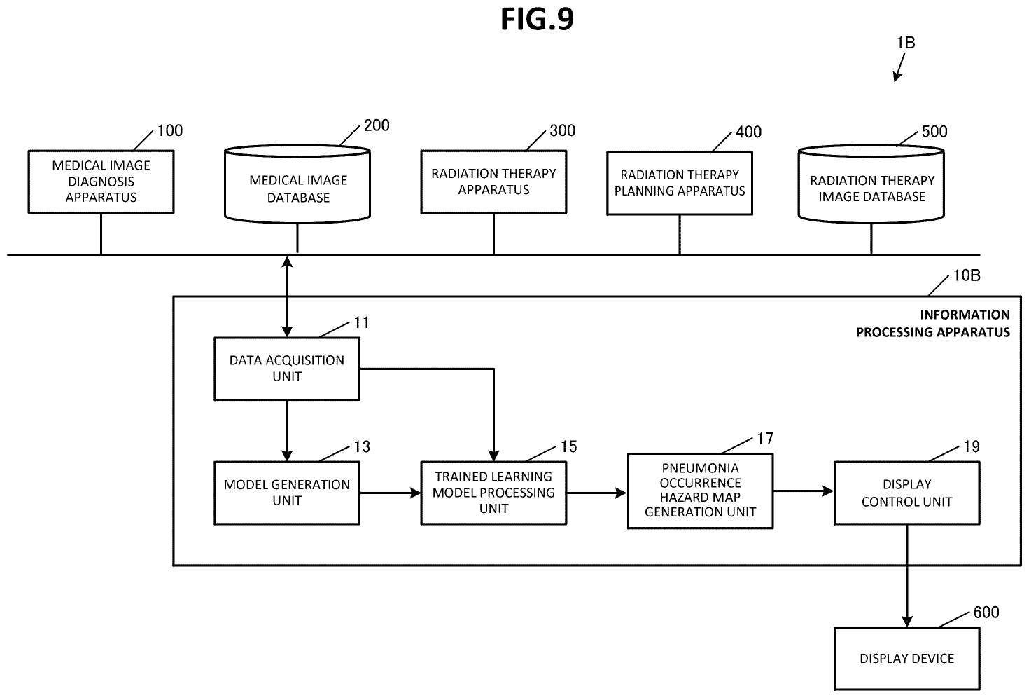

FIG. 9 is a diagram illustrating an example of a configuration of an information processing system 1B capable of implementing generation and output (display and the like) of the above-described pneumonia occurrence hazard map.

The information processing system 1B includes, for example, the medical image diagnosis apparatus 100, the medical image database 200, the radiation therapy apparatus 300, the radiation therapy planning apparatus 400, the radiation therapy image database 500, an information processing apparatus 10B as an example of the information processing apparatus, and the display device 600.

In the information processing system 1B, the information processing apparatus 10B includes, in addition to the data acquisition unit 11, the model generation unit 13, and the trained learning model processing unit 15, for example, a pneumonia occurrence hazard map generation unit 17 and a display control unit 19.

For example, the pneumonia occurrence hazard map generation unit 17 generates a pneumonia occurrence hazard map pneumonia occurrence based on an inferred susceptibility level output from the trained learning model processing unit 15.

The pneumonia occurrence hazard map generation unit 17 may be referred to as a functional unit configured to generate data for visualizing inferred pneumonia occurrence susceptibility levels as a map.

The display control unit 19 performs control of displaying, on the display device 600, the pneumonia occurrence hazard map generated by the pneumonia occurrence hazard map generation unit 17.

The display device 600 may be an output device (display device) configured to display various types of information according to the control of the display control unit 19.

The display device 600 may be, for example, a component of an apparatus including the information processing apparatus 10B. For example, the display device 600 may be a component (display unit) of the radiation therapy planning apparatus 400. The invention is not limited thereto, and the display device 600 may be a component (display unit) of an apparatus other than the radiation therapy planning apparatus 400.

The display control unit 19 may be configured as a functional unit of an apparatus including the display device 600. In this case, for example, the display control unit 19 may cause the display unit to display a pneumonia occurrence hazard map received from the information processing apparatus 10B by a communication unit (not illustrated).

The display device 600 may be a component of the information processing apparatus 10B.

In the information processing apparatus 10A illustrated in FIG. 1, the display control unit 19 may be provided at a stage subsequent to the trained learning model processing unit 15, and in the information processing system 1A illustrated in FIG. 1, the display device 600 that displays information in accordance with control of the display control unit 19 may be provided. Then, the display control unit 19 may perform control of displaying, on the display device 600, an inferred pneumonia occurrence susceptibility level output from the trained learning model processing unit 15.

### Processing

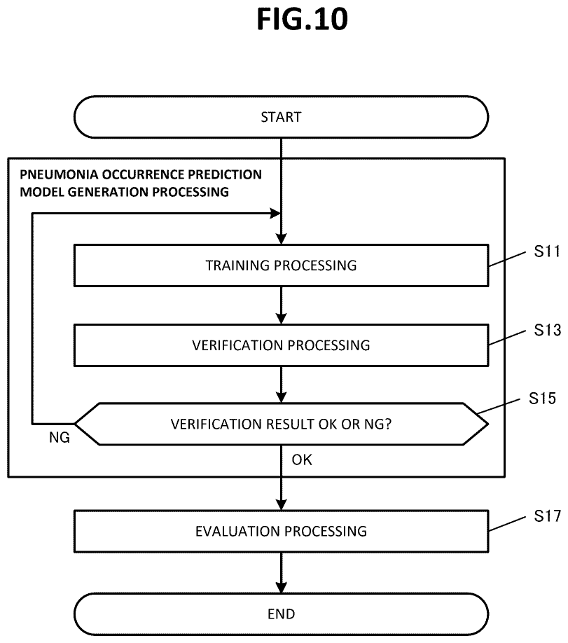

FIG. 10 is a flowchart illustrating an example of a procedure of information processing according to the embodiment, and is a flowchart illustrating an example of a flow of processing related to generation, verification, and evaluation of a model. The processing in this flowchart may be implemented by, for example, a processing unit (control unit) of the information processing apparatus 10B reading a program stored in a storage unit (not illustrated) into a RAM (not illustrated) and executing the program.

Hereinafter, each symbol S in the flowchart means a step.

The flowchart described below is merely an example of the procedure of the information processing according to the embodiment, and other steps may be added or some steps may be deleted. The flowchart may be executed with some steps in the flowchart replaced.

First, the model generation unit 13 performs pneumonia occurrence prediction model generation processing.

In the pneumonia occurrence prediction model generation processing, the model generation unit 13 performs training processing (S11). Specifically, for example, a plurality of examinees are divided into three groups (first group to third group), and a data set of examinees in the first group is used as a training data set, and a model is trained by the method described above.

Next, the model generation unit 13 performs verification processing of verifying the generated model (S13). Specifically, for example, the generated pneumonia occurrence prediction model is verified according to a predetermined verification method (verification algorithm) by using a data set of examinees in the second group as a verification data set.

Next, the model generation unit 13 determines which of OK and NG is the verification result (S15), and returns the processing to S11 if the verification result is NG (S15: NG). In this case, as an example, re-training may be performed by changing the first group.

On the other hand, if the verification result is OK (S15: OK), the model generation unit 13 ends the pneumonia occurrence prediction model generation processing.

Next, the processing unit performs evaluation processing of evaluating the generated model (S17). Specifically, for example, the generated pneumonia occurrence prediction model is evaluated according to a predetermined evaluation method (evaluation algorithm) by using a data set of examinees in the third group as a test data set. As the evaluation method, for example, cross-validation may be used.

The processing unit ends the processing.

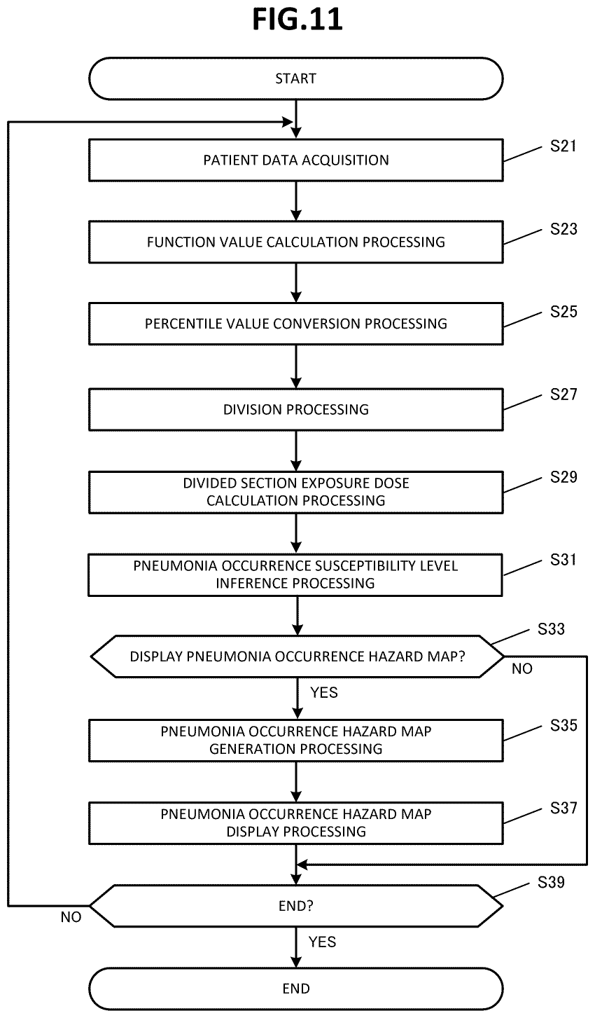

FIG. 11 is a flowchart illustrating an example of a procedure of the information processing according to the embodiment, and is a flowchart illustrating an example of a flow of processing related to prediction of pneumonia occurrence of a patient using the pneumonia occurrence prediction model generated in FIG. 10. The processing in this flowchart may be implemented by, for example, a processing unit (control unit) of the information processing apparatus 10B reading a program stored in a storage unit (not illustrated) into a RAM (not illustrated) and executing the program.

First, the data acquisition unit 11 acquires data regarding a living body (organ) of a patient (an example of a first examinee) to be diagnosed or treated (S21).

Next, the function value calculation unit 131 performs function value calculation processing of calculating a function value based on the data acquired in S21 (S23).

Thereafter, the percentile value conversion unit 133 performs percentile value conversion processing of converting the function value calculated in S23 into a percentile value (S25).

Next, the division unit 135 performs division processing of dividing the percentile value converted in S25 (S27).

Next, the divided section exposure dose calculation unit 137 performs divided section exposure dose calculation processing of calculating a divided section exposure dose for each divided section obtained in S27 (S29).

Thereafter, the trained pneumonia occurrence prediction model processing unit 159 performs pneumonia occurrence susceptibility level inference processing of inferring a pneumonia occurrence susceptibility level according to formula (2) based on the divided section exposure dose calculated in S29 and a stored model parameter (regression coefficient) (S31).

Next, the processing unit determines whether to display a pneumonia occurrence hazard map (S33). If it is determined to display a pneumonia occurrence hazard map (S33: YES), the pneumonia occurrence hazard map generation unit 17 performs pneumonia occurrence hazard map generation processing of generating a pneumonia occurrence hazard map (S35).

Then, the display control unit 19 performs control of displaying, on the display device 600, the pneumonia occurrence hazard map generated in S35 (S37).

The pneumonia occurrence hazard map may be generated as a two-dimensional map or may be generated as a three-dimensional map.

Various maps may be switched and displayed based on a user input.

Thereafter, the processing unit determines whether to end the processing (S39). If it is determined to continue the processing (S39: NO), the processing is returned to S21.

On the other hand, if it is determined to end the processing (S39: YES), the processing unit ends the processing.

Note that processing other than the processing of the generation of the pneumonia occurrence hazard map and the display control may be performed by the processing unit (control unit) of the information processing apparatus 10A illustrated in FIG. 1.

### Evaluation Result of Model

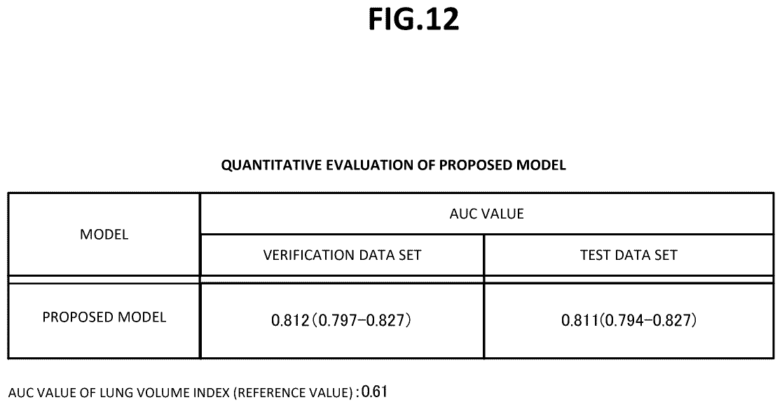

FIG. 12 is a diagram illustrating an example of an evaluation result of a generated model.

The inventors of the present application evaluate, by using nested 5-fold cross-validation, the generated pneumonia occurrence prediction model by using data sets of a certain number of patients of advanced lung cancer cases who are treated at the hospital of the present applicant and a cooperative medical institution.

Here, a case where a value of area under curve (hereinafter, referred to as “AUC value”) related to a function (dose function) of the exposure dose corresponding to the divided section is used as an evaluation index will be described. The AUC value is a value in a numerical range of “0 to 1”, and a larger value indicates a higher discriminative ability.

An average AUC value (reference value) calculated based on a method of simply reducing an exposed lung volume, which was performed by the inventors of the present application, was about “0.61”. On the other hand, in the test data set independent of the training data set and the verification data set, an average AUC value calculated based on the pneumonia occurrence prediction model generated by the method of the embodiment was about “0.81”, and it was confirmed that a high discriminative ability was indicated.

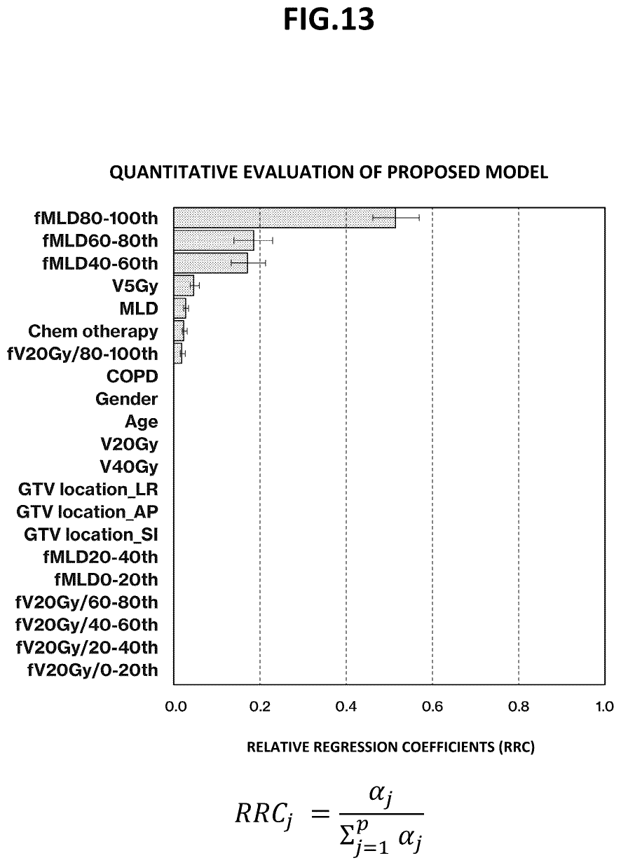

FIG. 13 illustrates an example of a table regarding the generated pneumonia occurrence prediction model and having a horizontal axis indicating relative regression coefficients (RRC) and a vertical axis indicating various values.

The relative regression coefficient RRC on the horizontal axis is an index value indicating a relative value of the above-described regression coefficient “α”, and is calculated according to, for example, the following formula (3).

$${RRC}_{j} = \frac{{α}_{j}}{{\sum }_{j = 1}^{p} {α}_{j}} \qquad ( 3 )$$

In the formula, the subscript “j” represents the number of the divided section (“j=1 to 5” in the above example), and “p” represents the total number of the divided sections (“5” in the above example).

On the vertical axis, “fMLD 80-100th”, “fMLD 60-80th”, “fMLD 40-60th”, “fMLD 20-40th”, and “fMLD 0-20th” correspond to the above-described divided sections “80-100th”, “60-80th”, “40-60th”, “20-40th”, and “0-20th”, respectively.

As described above, in the method focusing on the lung function, there is a problem that a plurality of functional regions cannot be used in an integrated manner.

On the other hand, in the method of the embodiment, it can be seen that the relative regression coefficient RRC of the divided section “80-100th” corresponding to a high-functioning region is the highest value of about “0.5”, the relative regression coefficient RRC corresponding to the divided section “60-80th” and the relative regression coefficient RRC corresponding to the divided section “40-60th” are also large values, and information on regions other than the high-functioning region is also used.

On the other hand, it can be seen that the relative regression coefficients RRC (regression coefficients α) of the divided section “0-20th” and the divided section “20-40th” are “0” (the divided section exposure dose “D₄” (20-40th) and the divided section exposure dose “D₅” (0-20th) are excluded) by the feature selection by the Lasso regression, and the information thereof is not used.

When another regression analysis method (described above) is used, the divided section exposure dose “D₄” (20-40th) and the divided section exposure dose “D₅” (0-20th) may affect the result.

### Application Example

In the method of the embodiment, it is possible to predict the occurrence of pneumonia accompanying radiation therapy, for example, non-invasively in an immediate manner based on an inferred pneumonia occurrence susceptibility level (non-invasiveness, immediacy). For example, it is possible to easily predict the occurrence of pneumonia based on information on a living body of the patient, without requiring the use of a special medical drug, without requiring to wait until the medical drug sufficiently takes effect, or without requiring any additional examination.

In addition, the method of the embodiment basically does not require additional equipment, and can be introduced as it is for existing radiation therapy.

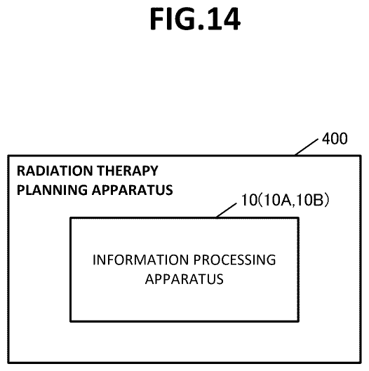

The information processing apparatus 10 of the embodiment may be incorporated in the radiation therapy planning apparatus 400 as illustrated in FIG. 14, for example.

The information processing apparatus 10 of the embodiment may be provided as a separate apparatus capable of communicating with the radiation therapy planning apparatus 400. In this case, the radiation therapy planning apparatus 400 may receive information from the information processing apparatus 10 by, for example, a communication unit.

The radiation therapy planning apparatus 400 may perform processing related to a radiation therapy plan (various types of processing related to the radiation therapy plan).

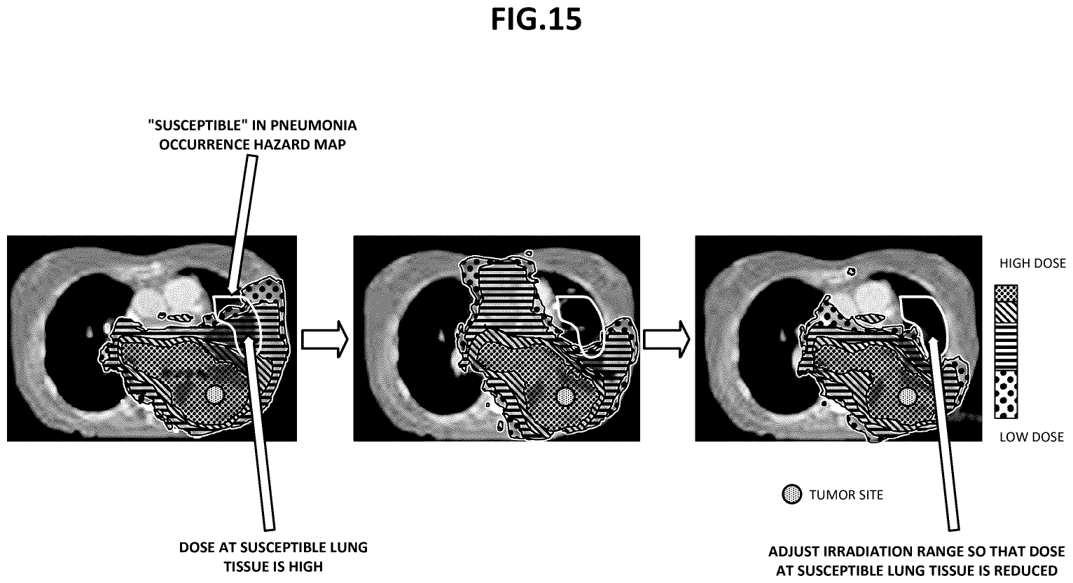

FIG. 15 is a diagram illustrating a radiation therapy plan, in which the images of IMRT in FIG. 2 are exemplified.

A left portion of the drawing shows an example of an image of the radiation therapy plan generated by the radiation therapy planning apparatus 400. Although the illustration of the pneumonia occurrence hazard map is omitted, facing the drawing, a region surrounded by a white line on the right side in the lung region is a “susceptible” region in the pneumonia occurrence hazard map, and the dose of some of them is high. That is, some of the dose of a susceptible lung tissue is high.

Therefore, it is conceivable that, as one method, the user performs an operation to adjust the irradiation range.

Specifically, the radiation therapy planning apparatus 400 displays, for example, a pneumonia occurrence hazard map and an image of the radiation therapy plan on the display unit. These images may be displayed on the same screen or may be displayed on different screens. The pneumonia occurrence hazard map may be displayed on the display device 600 separately.

Then, the user may operate an operation portion while viewing the pneumonia occurrence hazard map to adjust the irradiation range so that the dose of the susceptible lung tissue decreases (from a center portion to a right portion in the drawing). That is, the user may adjust the irradiation range based on the image of the radiation therapy plan generated by the radiation therapy planning apparatus 400.

Alternatively, the radiation therapy planning apparatus 400 may set the irradiation range based on the inferred pneumonia occurrence susceptibility level to generate an image of the radiation therapy plan.

Specifically, the processing unit (control unit) of the radiation therapy planning apparatus 400 may set the irradiation range such that, for example, the dose at a position at which the inferred pneumonia occurrence susceptibility level exceeds a threshold or is equal to or greater than the threshold is relatively lower than the dose at other positions. The threshold may be set, for example, to a value used for classifying into “susceptible” in the pneumonia occurrence hazard map.

The irradiation range may be further adjusted by the user based on the image of the radiation therapy plan generated in this manner.

In the conventional radiation therapy, a lung tissue highly susceptible to occurrence of pneumonia also receives a large amount of exposure.

In contrast, with the method of the embodiment, it is possible to reduce the exposure of the lung tissue highly susceptible to occurrence of pneumonia, based on an inferred pneumonia occurrence susceptibility level or a pneumonia occurrence hazard map.

In addition to the above, the processing unit of the radiation therapy planning apparatus 400 may perform processing of changing, for example, an inferred pneumonia occurrence susceptibility level.

In this case, the processing unit may perform the following processing, for example.
Determining a specific site based on volume data or the like.
Changing an pneumonia occurrence inferred susceptibility level of a site other than the specific site to a value that does not exceed the threshold (or a value less than the threshold).

For example, the specific site may include a site (hereinafter referred to as a “target site”) to be treated by the radiation therapy in the lung region, and may be set as a site within a predetermined range in the vicinity. The user may be able to set the extent of the predetermined range in the vicinity.

In this case,
regarding a site within a predetermined range in the vicinity, the dose can be set relatively high at a position where the inferred pneumonia occurrence susceptibility level does not exceed the threshold, and the dose can be set relatively low at a position where the inferred pneumonia occurrence susceptibility level exceeds the threshold, and
regarding the other sites (including a site of an organ other than the lungs), the dose can be set relatively high since the inferred pneumonia occurrence susceptibility level thereof does not exceed the threshold.

Basically, it is necessary to apply radiation to a tumor, but a position highly susceptible to the radiation may exist around the tumor, and in this case, the dose at the position can be set relatively low.

However, in this case, since a part of an organ other than the lungs falls within the irradiation range, a unique side effect may occur in the organ.

In general, a side effect of the radiation therapy tends to occur at a site where the radiation is applied. The side effect may occur during treatment or immediately after treatment, and may occur after elapse of a certain period of time or more. That is, when applying radiation, a normal organ may be affected. Therefore, for example, there is an approach not applying radiation to a normal organ (particularly, an important organ) different from an organ in which a tumor exists.

On the other hand, in case of a position where there is low susceptibility to occurrence of a side effect, it may be considered to apply radiation to a different organ.

In this case, for example, based on the method of the embodiment, a susceptibility level (an example of the first value related to the side effect of the radiation therapy) similar to the pneumonia occurrence susceptibility level may be inferred for a part or the whole of a different organ, and the same processing may be performed using not only the pneumonia occurrence susceptibility level but also the susceptibility level inferred for the different organ.

For example, the susceptibility level of a different organ may be inferred by quantifying function information based on morphological information acquired for the organ and performing the same processing as described above. When the function information can be directly acquired by nuclear medicine examination or the like, the function information may be used.

Similarly to the case of the pneumonia occurrence hazard map, a side effect occurrence hazard map indicating susceptibility to occurrence of a side effect in the different organ may be generated and output based on the inferred susceptibility level of the organ.

As the approach, there are roughly two approaches, that is,
an approach of including at least a part of the different organ in the irradiation range, and
an approach of not including the different organ in the irradiation range.

Similarly to the above, the processing unit may change the inferred susceptibility level based on, for example, the inferred pneumonia occurrence susceptibility level, the susceptibility level inferred for the different organ, and a corresponding position.

In this case, the processing unit may perform the following processing, for example.
Determining a specific site based on volume data or the like.
Changing a susceptibility level inferred for a site other than the specific site to a value that does not exceed a threshold (or a value less than the threshold).

The specific site may be, for example, a site within a predetermined range in the vicinity and a site of a specific organ different from the lungs. In this case,
regarding a site within a predetermined range in the vicinity and a site of a specific organ different from the lungs, the dose can be set relatively high at a position where inferred the pneumonia occurrence susceptibility level does not exceed the threshold, and the dose can be set relatively low at a position where the inferred pneumonia occurrence susceptibility level exceeds the threshold, and
regarding the other sites, the dose can be set relatively high since the inferred pneumonia occurrence susceptibility level thereof does not exceed the threshold.

Regarding the specific organ, the dose can be set relatively low at a position where the susceptibility level exceeds the threshold, and thus the exposure at the position highly susceptible to occurrence of a side effect can be reduced. For example, if the specific organ is an important organ, it is possible to reduce the exposure at a position highly susceptible to occurrence of a side effect in the important organ.

In case of the approach of not including a different organ in the irradiation range, the processing unit may change the inferred susceptibility level to a value exceeding the threshold, for example, for the specific organ. For example, if the specific organ is an important organ, the exposure of the important organ can be reduced without limitation.

In addition, the irradiation range may be set so as to avoid the specific organ, without inferring the susceptibility level regardless of the inferred susceptibility level.

These practices may be regarded as a method based on an approach of avoiding applying radiation to a specific organ.

For example, the processing unit may perform setting such that, for an organ among different organs which is considered to have a low possibility of occurrence of a side effect or in which the side effect poses a low risk even when occurring, at least a part of the organ falls within the irradiation range.

An approach of avoiding applying radiation to a specific site in an organ including a target site can be adopted. In this case, for example, the processing unit may change the inferred susceptibility level to a value not exceeding the threshold in the organ including the target site, except for a site within a predetermined range in the vicinity.

In this case, in the organ including the target site,
regarding a site within the predetermined range in the vicinity, the dose can be set relatively high at a position where the inferred susceptibility level does not exceed the threshold, and the dose can be set relatively low at a position where the inferred susceptibility level exceeds the threshold, and
regarding the other sites, the dose can be set relatively high since the inferred pneumonia occurrence susceptibility level thereof does not exceed the threshold.

### System Configuration and the Like

The method of the embodiment may be implemented by two or more apparatuses. For example, a system may be configured including a first information processing apparatus configured to generate a pneumonia occurrence prediction model, and a second information processing apparatus configured to infer a pneumonia occurrence susceptibility level using the pneumonia occurrence prediction model generated by the first information processing apparatus. For example, a system may be configured including the second information processing apparatus configured to infer a pneumonia occurrence susceptibility level, and a third information processing apparatus configured to generate a pneumonia occurrence hazard map based on the pneumonia occurrence susceptibility level inferred by the second information processing apparatus. That is, the method of the embodiment may be implemented by a system including two or more apparatuses.

Further, the information processing apparatus may infer the pneumonia occurrence susceptibility level based on, for example, a pneumonia occurrence prediction model that is generated in advance and stored in a storage medium or the like.

For example, the information processing apparatus may generate a pneumonia occurrence prediction model in accordance with a pneumonia occurrence prediction model generation program stored in advance in a storage medium or the like. The same applies to generation and display of the pneumonia occurrence hazard map, setting related to the radiation therapy plan, and the like.

### Mathematical Information

In the embodiment, it is sufficient that original information used for performing the processing is any mathematical information on an organ of a subject, and it does not matter what part of the organ the information represents. The mathematical information may include, for example, morphological information and function information.

The mathematical information may be, for example, information expressed on a real space relating to the organ of the subject (three-dimensional information, four-dimensional information when the time axis is taken into consideration). The mathematical information may be information expressed on a real plane (two-dimensional information).

The mathematical information may be acquired by any method. Although a dedicated device may be used, the mathematical information may be acquired without using the dedicated device.

For example, 2D information may be converted into pseudo 3D information to acquire the morphological information such as 3D CT information.

In addition to the method described in the embodiment, the function information may be acquired using a deep learning model such h as a convolutional neural network (CNN). For example, a technique of generating function information from 3D CT information using a deep learning model is disclosed in “A deep learning method for translating 3DCT to SPECT ventilation imaging: First comparison with 81mKr-gas SPECT ventilation imaging” (https://doi.org/10.1002/mp.15697), and this technique may be applied.

A technique using a generative adversarial network (GAN), a diffusion model, or the like may be applied.

The mathematical information may be acquired using different types of information.

For example, the mathematical information may be acquired using function information acquired by any method and morphological information acquired by any method. For example, a deep learning model that outputs mathematical information by taking function information and morphological information as inputs is generated. In this case, for example, if a model is trained to place more importance on the function information than the morphological information, it is possible to output information that does not ostensibly represent a function but appears to represent a function. That is, it is possible to acquire information that can be regarded (treated) as function information although not being function information ostensibly. For example, the information acquired in this manner may be used as function information to perform the above processing.

### Operation and Effect of Embodiment

In the embodiment, the information processing apparatus 10 includes the trained learning model processing unit 15 (an example of a calculation unit) configured to calculate a pneumonia occurrence susceptibility level (an example of a first value) corresponding to position information based on a pneumonia occurrence prediction model (an example of a model) and a predetermined value (an example of a second value), the pneumonia occurrence prediction model being generated using training data regarding a plurality of examinees (an example of a subject) and configured to calculate a pneumonia occurrence susceptibility level (an example of a first value regarding a side effect of radiation therapy), the predetermined value being based on information (an example of mathematical information regarding an organ and corresponding to the position information) such as a lung function value corresponding to position information of a lung (an example of the organ) of a first examinee (an example of a first subject).

Accordingly, the first value corresponding to the position information can be calculated based on the model and the second value, the model being generated using training data regarding a plurality of subjects and configured to calculate the first value regarding a side effect in the case of performing radiation therapy, the second value being based on the mathematical information regarding the organ of the first subject and corresponding to the position information. As a result, it is possible to appropriately support diagnosis, treatment, and the like in consideration of, for example, the side effect of radiation therapy by using the calculated first value.

The model may be generated by, for example, training of a model by machine learning using training data.

The model training may include, for example, training using one piece of training data and training (retraining) using another piece of training data based on a verification result of a model using verification data.

The function information, which is an example of the mathematical information, may be acquired, for example, based on morphological information (information on an anatomical structure) regarding an organ of the first examinee (an example of the first subject) and corresponding to the position information.

Accordingly, the function information can be easily and appropriately acquired based on the morphological information regarding the organ of the first subject and corresponding to the position information.

The predetermined value (an example of the second value) may be an exposure dose based on the mathematical information and exposure information regarding the radiation therapy.

Accordingly, the first value corresponding to the position information can be appropriately calculated based on the exposure dose and the model, the exposure dose being based on the mathematical information and the exposure information regarding the radiation therapy, and the model being generated using training data regarding the plurality of subjects and configured to calculate the first value regarding the side effect in the case of performing the radiation therapy.

The exposure dose may include a plurality of exposure doses divided based on the mathematical information.

Accordingly, it is possible to appropriately generate, by using training data including the plurality of exposure doses divided based on the mathematical information, a model for calculating the first value regarding the side effect of the radiation therapy.

The training data may be a set of data including a pneumonia occurrence susceptibility level (an example of the first value) and a plurality of exposure doses.

Accordingly, it is possible to generate, by using the set of data including the first value and the plurality of exposure doses, a model for calculating the first value regarding the side effect of the radiation therapy.

The model may be a pneumonia occurrence prediction model (an example of the model) represented by a weighted sum of the plurality of exposure doses.

Accordingly, the first value corresponding to the position information can be appropriately calculated using the model represented by the weighted sum of the plurality of exposure doses.

The information processing apparatus 10 may include the pneumonia occurrence hazard map generation unit 17 (an example of an image generation unit) configured to generate a pneumonia occurrence hazard map (an example of an image regarding the side effect) based on the position information and the pneumonia occurrence susceptibility level (an example of the first value) calculated using the pneumonia occurrence prediction model (an example of the model).

Accordingly, it is possible to generate an image regarding the side effect based on the position information and the first value calculated using the model. As a result, it is possible to appropriately support diagnosis, treatment, and the like by visualizing the image regarding the side effect.

The information processing apparatus 10 may include the display control unit 19 configured to perform control of displaying, on a display device, the pneumonia occurrence hazard map generated by the pneumonia occurrence hazard map generation unit 17.

Accordingly, it is possible to display the image regarding the side effect on the display device and allow a user to confirm the image.

The display device 600 may be provided including a display unit configured to display the pneumonia occurrence hazard map acquired from the information processing apparatus 10.

Accordingly, it is possible for the user to confirm the image regarding the side effect.

The radiation therapy planning apparatus 400 may be provided including the information processing apparatus 10 and a display unit configured to display the pneumonia occurrence hazard map and an image of the lung (an image of a radiation therapy plan) with which an irradiation range of radiation in the radiation therapy is associated.

Accordingly, the user can compare the image regarding the side effect with the image of the radiation therapy plan.

In this case, the irradiation range may be changeable based on a user operation.

Accordingly, it is possible for the user to adjust the irradiation range of radiation while viewing the image regarding the side effect.

The radiation therapy planning apparatus 400 may be provided including a processing unit configured to perform processing related to planning of radiation therapy based on the position information, which is acquired from the information processing apparatus 10 and is corresponding to the mathematical information, and the pneumonia occurrence susceptibility level (an example of the first value) calculated using the pneumonia occurrence prediction model (an example of the model).

Accordingly, it is possible to provide a radiation therapy planning apparatus that performs processing related to planning of radiation therapy based on the position information, which is acquired from the information processing apparatus and is corresponding to the mathematical information, and the first value calculated using the model.

The pneumonia occurrence susceptibility level may be a value of which a larger magnitude indicates higher susceptibility to the side effect. The processing unit may set the irradiation range of radiation such that a dose at a position at which an inferred pneumonia occurrence susceptibility level exceeds a threshold or is equal to or greater than the threshold is relatively lower than a dose at other positions.

Accordingly, it is possible to set the irradiation range to reduce the exposure at a position where the susceptibility to occurrence of the side effect is high.

Further, the pneumonia occurrence susceptibility level may be a value of which a larger magnitude indicates higher susceptibility to the side effect. The processing unit may change inferred pneumonia occurrence susceptibility level so that the inferred pneumonia occurrence susceptibility level does not exceed a threshold or is not equal to or greater than the threshold, except for a specific site.

Accordingly, for example, the dose at a position excluding the specific site can be set relatively high.

In this case, the specific site may include at least a site within a predetermined range in the vicinity of a target site of the radiation therapy.

Accordingly, for example, the dose at a position, which excludes at least the site within the predetermined range in the vicinity of the target site of the radiation therapy, can be set relatively high.

In this case, the specific site may include a site of a specific organ different from an organ including the target site, in addition to the site within the predetermined range in the vicinity described above.

Accordingly, for example, the dose at a position, which excludes the site within the predetermined range in the vicinity including the target site of the radiation therapy and excludes the site of the specific organ, can be set relatively high.

Further, the pneumonia occurrence susceptibility level is a value of which a larger magnitude indicates higher susceptibility to the side effect. The processing unit may change the inferred pneumonia occurrence susceptibility level so that the inferred pneumonia occurrence susceptibility level does not exceed a threshold or is not equal to or greater than the threshold in the organ including the target site of the radiation therapy, except for the site within the predetermined range in the vicinity including the target site.

Accordingly, for example, in the organ including the target site of the radiation therapy, the dose at a position excluding the site within the predetermined range in the vicinity can be set relatively high.

### First Modification of Embodiment

The method of the above-described embodiment may be similarly applied to medical practices such as examination and diagnosis using radiation in addition to radiation therapy. That is, the method may be applied to all radiation therapy systems including treatment, examination, diagnosis, and the like.

Further, the method of the above-described embodiment is not limited to the treatment of lung cancer, and can be similarly applied to a practice as long as the practice performs treatment involving exposure.

The subject is not limited to a human and may be an animal (excluding a human). In this case, the subjects are of the same kind. For example, if the subject is a dog, a susceptibility level may be calculated using a model generated using training data regarding a plurality of dogs, and if the subject is a cat, a susceptibility level may be calculated using a model generated using training data regarding a plurality of cats.

When performing radiation therapy for cancer or the like of an organ other than the lungs, a susceptibility level similar to the pneumonia occurrence susceptibility level may be inferred and output for the organ according to the same method as described above. A side effect occurrence hazard map may be generated and output for the organ based on the inferred susceptibility level.

Further, setting related to the above-described radiation therapy plan may be performed for the organ.

### Second Modification of Embodiment

For example, the pneumonia occurrence susceptibility level in the above-described embodiment may be defined and calculated as a value in a numerical range of “0 to 1” or “0 to 100”. In this case, the pneumonia occurrence susceptibility level may be a pneumonia occurrence susceptibility degree indicating the susceptibility to occurrence of pneumonia with the rate.

Further, the pneumonia occurrence susceptibility level may be defined and calculated as a value of which a smaller magnitude indicates higher susceptibility to occurrence of pneumonia.

Instead of the pneumonia occurrence susceptibility level, a pneumonia occurrence safety value (for example, a value of which a larger magnitude indicates a higher level of safety) representing the unlikelihood (safety) of occurrence of pneumonia may be defined and calculated. Then, a pneumonia safety map may be generated based on the pneumonia occurrence safety value.

For example, the pneumonia occurrence susceptibility level and the pneumonia occurrence safety value may be an example of the first value regarding the side effect of the radiation therapy. When any one of the two values is used, for example, occurrence and non-occurrence, susceptible and safe states, and the like can be determined by introducing a threshold (cutoff value).

The same applies to a case of performing processing for an organ other than the lungs.

### Third Modification of Embodiment

In the above-described embodiments, there are five divided sections, and the invention is not limited thereto.

The number of divided sections can be appropriately set, and may be set to “10”, “15”, or “20”, for example.

This setting may be automatically performed by the information processing apparatus (automatic setting) or may be set by the information processing apparatus based on a user input (manual setting).

Since information is insufficient if the division number is too small, the number may be a value of about “3” to “10”, for example. The inventors of the present application have found that a good result can be obtained when the division number is “5”, and therefore the description is made by setting the division number to “5” in the above-described embodiments.

The same applies to a case of performing processing for an organ other than the lungs.

What is claimed is:

1. An information processing apparatus comprising:
a calculation unit configured to calculate a first value corresponding to position information based on a model and a second value, the model being generated using training data regarding a plurality of subjects and configured to calculate the first value regarding a side effect of radiation therapy, the second value being based on mathematical information regarding an organ of a first subject and corresponding to the position information.

2. The information processing apparatus according to claim 1, wherein
the second value is an exposure dose based on the mathematical information and exposure information regarding the radiation therapy.

3. The information processing apparatus according to claim 2, wherein
the exposure dose is a plurality of exposure doses divided based on the mathematical information.

4. The information processing apparatus according to claim 3, wherein
the training data is a set of data including the first value and the plurality of exposure doses.

5. The information processing apparatus according to claim 4, wherein
the model is a model represented by a weighted sum of the plurality of exposure doses.

6. The information processing apparatus according to claim 1, further comprising:
an image generation unit configured to generate an image regarding the side effect based on the position information and the first value calculated by the calculation unit.

7. A display device comprising:
a display unit configured to display an image regarding the side effect acquired from the information processing apparatus according to claim 6.

8. A radiation therapy planning apparatus comprising:
the information processing apparatus according to claim 6; and
a display unit configured to display an image regarding the side effect and an image of the organ of the first subject with which an irradiation range of radiation in the radiation therapy is associated.

9. A radiation therapy planning apparatus comprising:
a processing unit configured to perform processing related to planning of the radiation therapy based on both the position information corresponding to the mathematical information and the first value calculated by the calculation unit, which are acquired from the information processing apparatus according to claim 1.

10. The radiation therapy planning apparatus according to claim 9, wherein
the first value is a value of which a larger magnitude indicates higher susceptibility to the side effect, and
the processing unit sets an irradiation range of radiation such that a dose at a position where the first value calculated by the calculation unit exceeds a threshold or is equal to or greater than the threshold is relatively lower than a dose at another position.

11. The radiation therapy planning apparatus according to claim 9, wherein
the first value is a value of which a larger magnitude indicates higher susceptibility to the side effect, and
the processing unit changes the first value calculated by the calculation unit so that the first value does not exceed a threshold or is not equal to or greater than the threshold, except for a specific site.

12. The radiation therapy planning apparatus according to claim 11, wherein
the specific site includes at least a site within a predetermined range in vicinity of a target site of the radiation therapy.

13. The radiation therapy planning apparatus according to claim 12, wherein
the specific site includes a site of a specific organ different from the organ.

14. The radiation therapy planning apparatus according to claim 9, wherein
the first value is a value of which a larger magnitude indicates higher susceptibility to the side effect, and
the processing unit changes the first value calculated by the calculation unit so that the first value does not exceed a threshold or is not equal to or greater than the threshold in the organ, except for a site within a predetermined range in vicinity of a target site of the radiation therapy.

15. An information processing method comprising:
calculating a first value corresponding to position information based on a model and a second value, the model being generated using training data regarding a plurality of subjects and configured to calculate the first value regarding a side effect of radiation therapy, the second value being based on mathematical information regarding an organ of a first subject and corresponding to the position information.

16. A program for causing a computer to execute:
calculating a first value corresponding to position information based on a model and a second value, the model being generated using training data regarding a plurality of subjects and configured to calculate the first value regarding a side effect of radiation therapy, the second value being based on mathematical information regarding an organ of a first subject and corresponding to the position information.
